(19) United States
(12) Patent Application Publication
(10) Pub. No.: US 2014/0031905 A1
(43) Pub. Date: Jan. 30, 2014

(54) BURST PULSE TISSUE STIMULATION METHOD AND APPARATUS

(71) Applicant: Purdue Research Foundation, West Lafayette, IN (US)

(72) Inventors: Pedro P. Irazoqui, Lafayette, IN (US); Matthew Peter Ward, Indianapolis, IN (US); Kurt Yuqin Qing, Carmel, IN (US)

(21) Appl. No.: 13/941,153

(22) Filed: Jul. 12, 2013

Related U.S. Application Data
(60) Provisional application No. 61/671,013, filed on Jul. 12, 2012.

(51) Int. Cl. A61N 1/36

(52) U.S. Cl. CPC A61N 1/36189
USPC 607/73

(57) ABSTRACT

A method for stimulating nerve tissue of an organism includes a step of electrically connecting an electrical signal source to at least a first nerve. The method also includes applying a periodically repeating burst pulse signal pattern to the first nerve. The burst pulse signal pattern has a pattern frequency defining a frequency of repetition of the burst pulse signal pattern and a pattern duty cycle defining a first time period of the burst pulse signal pattern, the burst pulse signal pattern having a plurality of pulses, the pulses having a frequency within the pattern that exceeds the pattern frequency by at least an order of magnitude.

The present application claims the benefit of U.S. Provisional Patent Application Ser. No. 61/671,013, filed Jul. 12, 2012, which is incorporated herein by reference.

### BACKGROUND

For over two centuries now, electrical stimulation has been used to modulate the activity of various human physiological systems, most notably the nervous system. In particular, it is known to provide electrical stimulation to various nerves via electrodes or terminals. Conventionally, the electrical stimuli are composed of pulses of electrical charge, either controlled by voltage or current. These controlled voltage and/or current pulses are applied to a patient at or near the location of one or more tissues, such as nerve tissue. A summary and comparison of the advantages and disadvantages of typical pulse waveforms can be found in Merrill D. R., Bikson M., and Jeffreys J. G., “Electrical Stimulation of Excitable Tissue: Designe of Efficacious and Safe Protocols” Journal of Neuroscience Methods, Vol. 141, pp. 171-198 (2005) (hereinafter “the Merrill Article” 2005).

For such pulse waveforms, the most common pulse shape used in research and clinical settings is the simple rectangle. Rectangular pulses have been used for many decades and have been proven safe, efficacious and easy to implement. While other pulse shapes have been attempted, the rectangular pulse remains the most common. A typical pulse waveform has a pulse frequency of 10 Hz to 30 Hz, meaning that the pulses repeat 10 to 30 times per second.

As illustrated in FIGS. 1a-1d, it is known to vary certain parameters of the pulse for the purpose of causing different biological effects. The pulses may be anodic (positive from zero) or cathodic (negative from zero). The pulses may be monophasic, meaning that the pulses are all cathodic or all anodic, or biphasic, wherein both types of pulses are present. FIG. 1a, for example, shows a monophasic cathodic pulse waveform. FIG. 1b, by contrast, shows a biphasic pulse waveform. In FIG. 1b, the biphasic pulse waveform is said to be balanced because the anodic and cathodic pulses have the same amplitude. By contrast, FIG. 1c shows an imbalanced biphasic pulse waveform. Biphasic pulse waveforms may or may not include delays, or parts that are at zero amplitude. FIGS. 1b and 1c show biphasic pulse trains without delays, and FIG. 1d shows a biphasic pulse train having delays. In general, cathode-leading waveforms have been widely shown to be more effective.

As noted above, the pulse waveforms shown in FIGS. 1a-1d are applied to a patient as repeating waveform having a repetition frequency of approximately 10 Hz to 30 Hz. FIG. 2 illustrates a sample stimulus waveform that is created using a train of biphasic, charge balanced, rectangular pulse waveforms with interphase delay. The waveforms can be characterized by parameters. The key parameters of such a stimulus waveform such as shown in FIG. 2 are as follows:
Cathodic amplitude (AMPc)—amplitude (either voltage or current) of the cathodic pulse
Anodic amplitude (AMPa)—amplitude (either voltage or current) of the anodic pulse
Pulse period (PP)—the time between the beginning of two successive pulses; this is equal to 1/PRF, the pulse repetition frequency (unit Hz)
Interpulse interval (IPI)—the time between the end of the first pulse and the beginning of the following pulse
Pulse width (PW)—the duration (a time value) of each pulse; the PW of the cathodic phase does not necessarily have to match that of the anodic
Interphase delay (IPD)—the delay (a time value) between cathodic and anodic phases; this value could be 0 but usually is not longer than the IPI
   The effect of altering all the parameters (for each pulse waveform and the pulse train) listed above has been studied, for example in the Merrill Article, as well as in Kuncel, A. M., and Grill, W. M. “Selection of Stimulus Parameters for Deep Brain Stimulation.” Clinical Neurophysiology, Vol. 115, pages 2431-41(2004). Overall, this paradigm of stimulation using electrical pulses has been shown widely to have physiological and clinically therapeutic effect and has long been established as a safe and effective.

In certain applications, more complex features are introduced into the stimulus waveform in the form of amplitude (AM) and/or frequency modulation (FM) of the pulse parameters within the pulse train. In AM, the amplitude parameters of the individual pulses within the train are varied; in FM, the time parameters are varied. For instance, in the field of cochlear implants, the advantages of AM and FM pulse trains have been characterized and are well-established. See, for example, Wilson, B. S. et al. “Better Speech Recognition With Cochlear Implants”, Nature, Vol. 352, pages 236-238 (1991).

Despite the reasonable success of these methods, there is always a need for identifying more efficient and/or efficacious methods of tissue stimulation via electrical signals.

### SUMMARY

At least some embodiments of the present invention address the above-described need, as well as others, by implementing tissue stimulus waveforms using “burst modulation”. This method uses brief burst pulses, much shorter than the standard pulse itself, to construct each pulse of the stimulus.

A first embodiment is a method for stimulating nerve tissue of an organism that includes a step of electrically connecting an electrical signal source to at least a first nerve. The method also includes applying a periodically repeating burst pulse signal pattern to the first nerve. The burst pulse signal pattern has a pattern frequency defining a frequency of repetition of the burst pulse signal pattern and a pattern duty cycle defining a first time period of the burst pulse signal pattern, the burst pulse signal pattern having a plurality of pulses, the pulses having a frequency within the pattern that exceeds the pattern frequency by at least an order of magnitude.

A second embodiment is a method for stimulating nerve tissue of an organism that similarly includes electrically connecting an electrical signal source to at least a first nerve. The method also includes applying a periodically repeating burst pulse signal pattern to the first nerve, the burst pulse signal pattern having a pattern frequency defining a frequency of repetition of the burst pulse signal pattern and a pattern duty cycle defining a first time period of the burst pulse signal pattern. The burst pulse signal pattern has a plurality of pulses, wherein applying the periodically repeating burst pulse signal pattern includes applying a select burst signal pattern corresponding to a type of the first nerve.

A third embodiment is a system for stimulating tissue that implements any of the above described methods.

In some embodiments, these discrete burst pulses occur at such a high rate that neurons would “perceive” the burst pulses as a continuous pulse.

The above described features and advantages, as well as others, will become more readily apparent to those of ordinary skill in the art by reference to the following detailed description and the accompanying drawings.

### BRIEF DESCRIPTION OF THE DRAWINGS

FIGS. 1a-1d show timing diagrams of examples of prior art pulse patterns for tissue stimulation;

FIG. 2 shows a timing diagram of an exemplary prior art pulse pattern waveform, the pulse pattern comprising a repeating pattern of biphasic, charge balanced, rectangular pulse waveforms with interphase delay;

FIG. 3 shows a schematic block diagram of an exemplary arrangement for providing pulse stimulation to nerve tissue according to a first embodiment of the invention;

FIG. 4 shows a timing diagram of an exemplary repeating pulse pattern according to a first embodiment of the invention;

FIG. 5 show a timing diagram of an exemplary pulse pattern illustrating the parameters by which the pulse pattern according to embodiments of the invention can be characterized;

FIGS. 6A and 6B show timing diagram of additional exemplary pulse patterns according to embodiments of the invention;

FIG. 7 shows a schematic block diagram of a first embodiment of a signal generator and processing circuit of the arrangement of FIG. 3;

FIG. 8 shows a schematic block diagram of a second embodiment of a signal generator and processing circuit of the arrangement of FIG. 3;

FIG. 9 shows charge-response curves of vagal C fibers resulting from vagus nerve stimulation in a rat using different stimulus waveforms;

FIG. 10 shows charge-response curves of vagal A and C fibers resulting from vagus nerve stimulation in a rat using different stimulus waveforms;

FIG. 11 shows compound action potential (CAP) recordings resulting from stimulation of the sciatic nerve in a rat.

### DETAILED DESCRIPTION

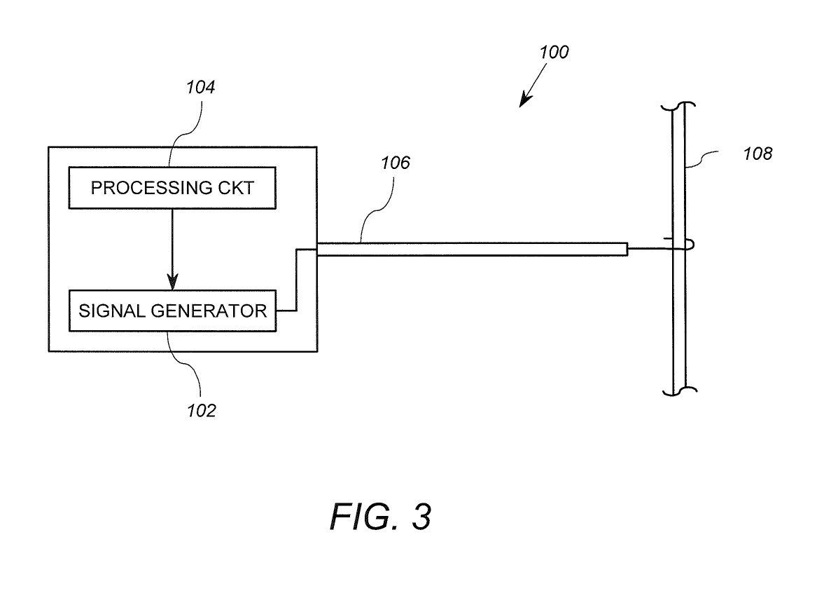

FIG. 3 shows a schematic block diagram of an exemplary arrangement 100 for providing pulse stimulation to nerve tissue according to a first embodiment of the invention. The arrangement includes a signal generator 102, a processing circuit 104 and at least one electrode 106. The at least one electrode 106 may be any conventional electrode for coupling pulse signals to tissue. Suitable electrodes for providing pulse signals to the Vagus nerve, the sciatic nerve, and others are well known in the art. In at least some embodiments, all or part of the arrangement 100 is all implantable in a living organism. In other cases, the signal generator 102 and processing circuit 104 are portable, and capable of being mounted on or supported by an ambulatory patient.

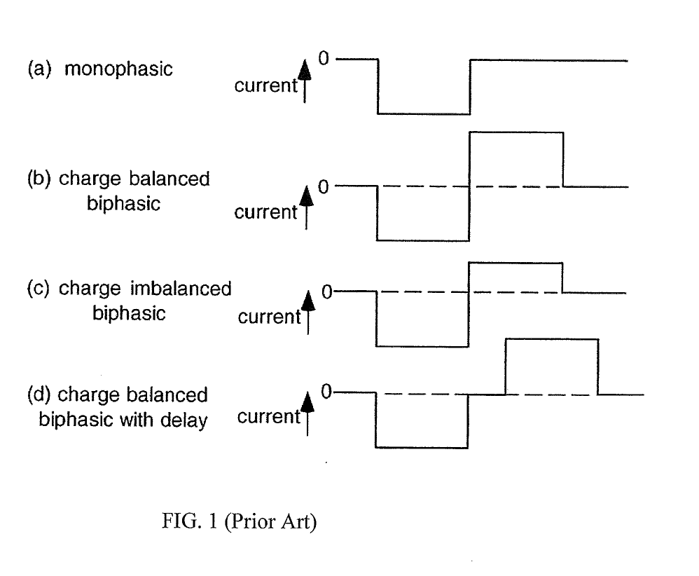

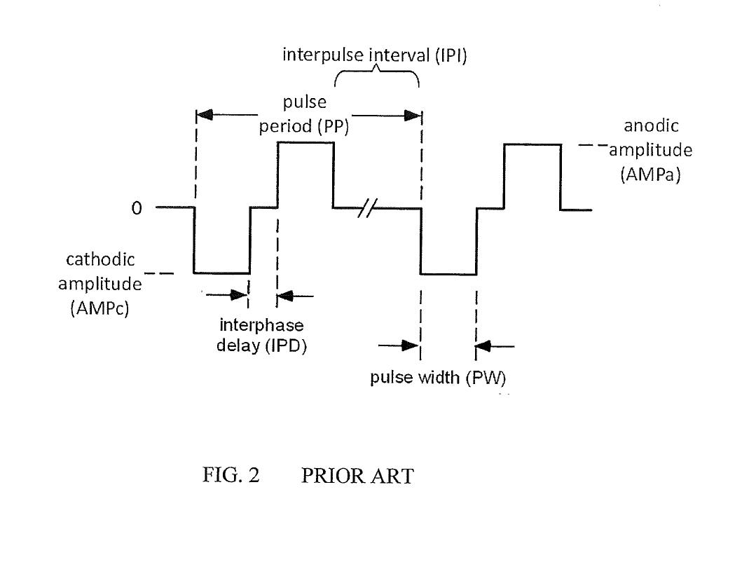

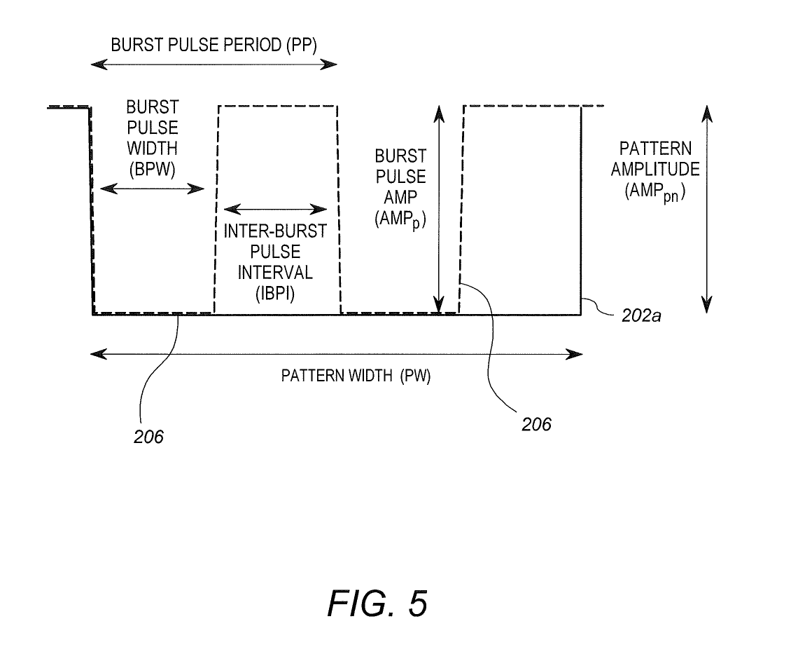

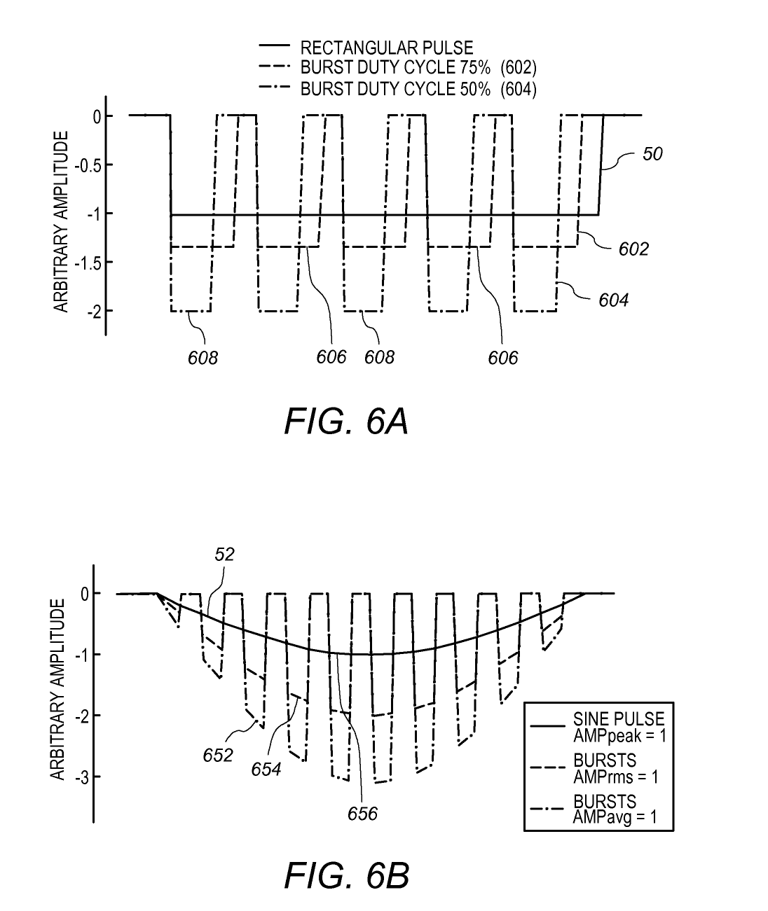

According to the exemplary embodiment of FIG. 1, the signal generator 102 is a pulse generator that is configured to generate pulses having a duration of less than 1 millisecond. As will be discussed below in detail in connection with FIGS. 5 and 6, the signal generator 102 is configured to generate either current pulses, voltage pulses, or both. The processing circuit 104 may suitably be a processing circuit that is housed in the same structure as the signal generator 102, and is configured to cooperate with the signal generator to generate a periodically repeating burst pulse signal pattern.

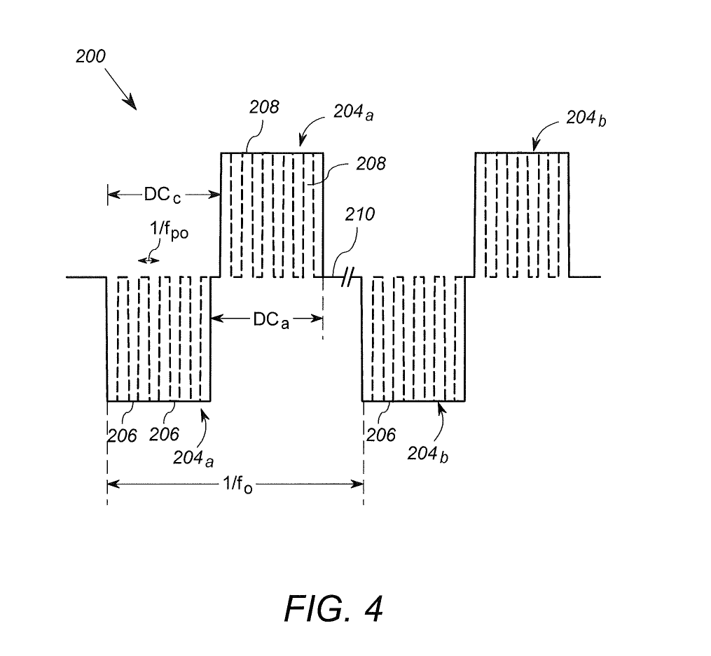

With reference to FIG. 4, an example of a waveform 200 that is produced by the signal generator 102 in accordance with at least one embodiment of the invention is shown. The waveform 200 comprises periodically repeating, alternating, cathodic and anodic burst pulse patterns 202, 204. The waveform 200 in this example is biphasic, including a first cathodic pulse signal pattern 202a, 202b, etc., that is interleaved with a second anodic pulse signal pattern 204a, 204b. The waveform 200 has a pattern frequency f₀ defining a frequency of repetition of the first pulse signal patterns 202a, 202b, which is the same as the frequency of repetition of the second pulse signal patterns 204a, 204b. Accordingly, as shown in FIG. 4, the pattern period 1/f₀ defines the time from the start of a first cathodic pulse signal pattern 202a and the start of the next cathodic pulse signal pattern 202b.

The cathodic pulse signal pattern 202a also has a pattern duty cycle, which is defined as the time length DC_{c} of the pattern 202a over the period 1/f₀. Similarly, the anodic pulse signal pattern 204a has a pattern duty cycle, which is defined as the time length DCₐ of the pattern 204a over the period 1/f₀. As shown in FIG. 4, the pulse patterns 202a, 204a each comprise a respective plurality of pulses 206, 208. The pulses 206, 208 having a pulse frequency fp₀ within their respective patterns 202a, 202b, 204a, 204b which is defined by the rate at which the burst pulses 206, 208 repeat within a specific pattern. In the exemplary embodiment disclosed herein, the pulse frequency fp₀ exceeds the pattern frequency f₀ by at least an order of magnitude. The waveform 200 also includes an inter-pattern delay 210 defined between the anodic pattern 204a and the subsequent cathodic pattern 202b.

Referring back to FIG. 3, the signal generator 102 is operably coupled to provide the signals to at least one electrode 106. The at least one electrode 106 in FIG. 3 is coupled to deliver the periodically repeating burst pulse signal pattern to a nerve 108.

This bursting paradigm shown in FIG. 4 introduces an entirely new dimension to electrical stimulation while still capable of maintaining the stimulus parameters of the conventional pulsing paradigm. In particular, the burst pulse signal pattern 202a, 202b, etc. can have varied attributes similar to those of the pulses of the prior art. In particular, each pattern 202a, 204a, etc. corresponds to a single prior art pulse such as those shown in FIG. 2. However, each pattern 202a, 204a etc. represents a single prior art pulse that has been broken up into high frequency burst pulses 206, 208. Thus, the pattern frequency, pattern shape, pattern duty cycle and even the inter-pattern delay correspond to the pulse frequency, pulse shape and inter-pulse delay of the prior art 10 Hz to 30 Hz pulse signals. In FIG. 4, the repeating burst pulse pattern waveform 200 defines a pattern signal that is cathode pattern leading (i.e. starting with cathode pattern 202a) and biphasic (alternating cathodic and anodic patterns 202a, 204a, 202b, 204b, etc.). The waveform 200 is charge-balanced (cathodic pulses 206 and anode pulses 208 have the same magnitude), and has an inter-pattern delay 210. It will be appreciated that other pattern signals can be monophasic, unbalanced, and varied in other ways prior art pulse signals were varied. However, as discussed above, each pattern instance 202a, 204a etc. is broken up into high frequency burst pulses.

Moreover, attributes of the burst pulses 206, 208 within the patterns 202a, 204a may also be altered to suit the type of tissue being stimulated. FIG. 5 shows in further detail an exemplary version of the pattern waveform 202a where the number of the pulses 206 in the pattern 202a is two instead of five, as shown in FIG. 4. As shown in FIG. 5, the new parameters of the burst pulse pattern 202a can include:
Pulse amplitude (AMPp)—the amplitude (either voltage or current) of each burst pulse 206.
Number of pulses (NOP)—the number of burst pulses 206 used to construct each longer burst pulse pattern 202a.
Burst pulse width (BPW)—the duration (a time value) of each burst pulse 206.
Inter-pulse interval (IBPI)—the duration between the end of the first burst pulse 206 and the beginning of the following burst pulse 206.
Burst pulse period (PP)—the duration of each burst pulse 206 including the “off” phase between the end of the pulse 206 and the beginning of the next pulse 206.
Burst pulse duty cycle (PDC)—the duty cycle of each burst pulse 206 (a fraction or percentage).
Pattern amplitude (AMPpn)—an defined amplitude of the pattern 202a that may be based on a prior art pulse amplitude such as that shown in FIG. 2.
   The PDC is a derived parameter, defined using the formula: PDC=BPW/PP. When assessing all the burst pulses, there is an extra parameter, “duty cycle within pattern” (DCP), which is defined as: DCP=BPW*NOP/PW. DCP accounts for the fraction of time within each pulse that stimulation is “on” and is a more accurate measure than PDC, because some combinations of NOP and PW do not yield a perfect fit.

In the example of FIG. 5, the pattern 202a is a cathodic rectangular pulse (gray solid line) constructed from two burst pulses 206 (black dashed line). Here, AMPp=the peak amplitude of the pattern AMPpn, NOP=2 (normally will exceed 5), PP=2*BPW=2*IBPI, and PDC=50%.

As shown in FIG. 5, the pattern amplitude AMPpn is merely equal to the (peak) pulse amplitude AMPp. However, in other embodiments, the amplitudes of the pulses may be selected such that the pattern amplitude AMPpn is the average of the amplitudes of all of the pulses 206 or the root mean square of the pulses 206. It will be appreciated that such a definition has significance when the pattern 202a is intended to adopt the overall shape and characteristics of a prior art pulse pattern. As discussed above, the waveforms 200 of at least some embodiments of the present invention are intended to implement patterns based on prior art pulse signals, wherein each prior art pulse replaced by a burst pulse pattern that represents the prior art pulse broken into the higher frequency burst pulses 206, 208.

Accordingly, starting from a prior art pulse train such as shown in FIGS. 1a-1d, which may have a defined prior art pulse amplitude, the user can develop a repeating burst pulse pattern using the defined pulse amplitude as the pattern amplitude AMPpn. As mentioned above, it is possible to set 1) the average, 2) the root mean squared, or 3) the peak amplitude of the burst modulated pulses equal to AMPpn. The DCP parameter is used for amplitude scaling calculations. Amplitude scaling option 1 (average) will match the overall electrical charge injected by each pulse, and option 2 (RMS) attempts to match the energy injected. It is important to note that for option 2, because the effective impedance of the stimulation system will be lower for burst modulated stimulation, the energy-matching feature is only approximate.

By way of illustrative example, FIG. 6A shows amplitude scaling matching to the average amplitude with a rectangular-shaped pulse, as well as bursting at different duty cycles. In particular, FIG. 6A shows an original prior art pulse 50, and two pulse patterns 602, 604. The pulse pattern 602 comprises five burst pulses 606 having a magnitude AMPp that is 33% greater than AMPpn, and a pulse duty cycle PDC of 75%. The pulse pattern 604 comprises five burst pulses 608 having a magnitude AMPp that is 100% greater than AMPpn, and a pulse duty cycle PDC of 50%.

FIG. 6B, on the other hand, shows a sine-shaped pulse demonstrating the three amplitude scaling options (average, RMS and peak) for the pulses 652, 654, 656 compared to the prior art pulse 52.

Referring again to FIG. 3, the signal generator 102 and the processor 104 may take many forms. In some embodiments, the waveforms according to the present invention may be in some cases carried out by manual settings on a suitable signal generator 102. In such a case, a processing circuit 104 may not be necessary. In other embodiments, a processor 104, which may or may not be part of the packaged system of the signal generator 102, provides the parameters and/or other control that cause the signal generator 102 to generate the burst pulse pattern waveforms.

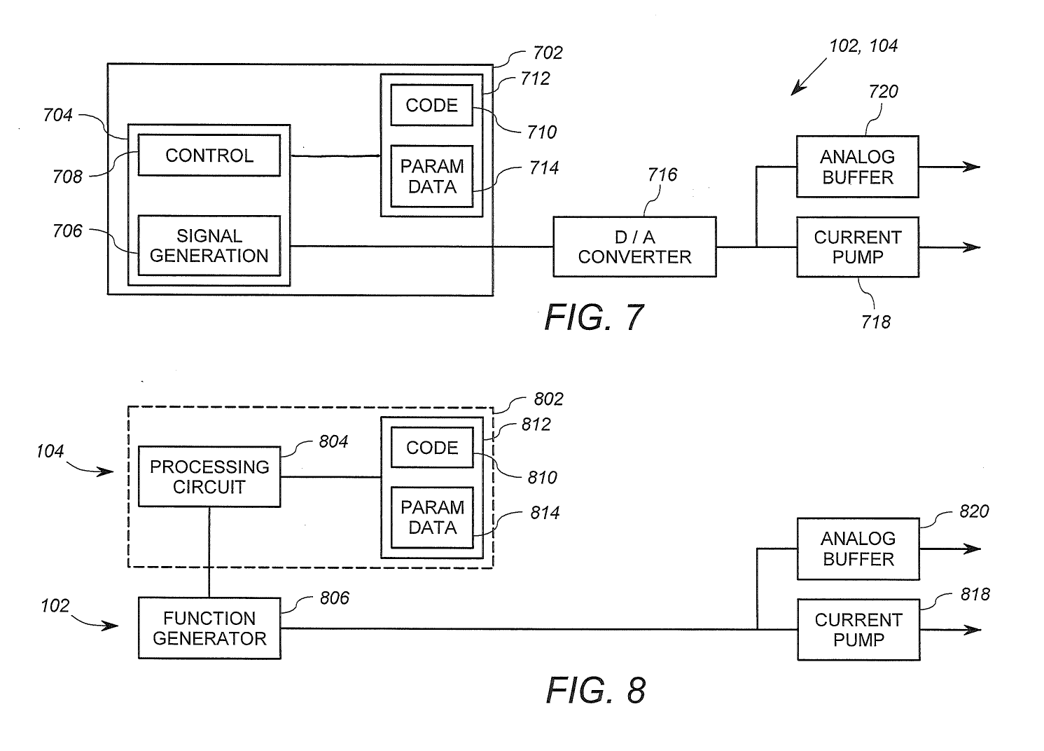

FIGS. 7 and 8 show two different examples of suitable embodiments of the signal generator 102 and the processing circuit 104 of FIG. 3. In FIG. 7, the signal generator and the processing circuit 102, 104 are both parts of a general purpose computer 702. The signal generator 102 further includes a digital-to-analog converter 716, a current pump 718, and an analog buffer or amplifier 720.

In FIG. 7, the computer 702 includes a processing circuit 704 that executes program instructions to carry out a signal generation function 706 and to carry out a control function 708. The instructions or code 710 for such functions are stored in a memory 712 of the computer 702. The memory 712 may also suitably store sets of pulse and/or pattern parameters that correspond specifically to different tissues (e.g. nerves) to be stimulated. For example, the parameter data 714 may include a plurality of sets of parameter values, and for each set of parameter values, an association with one or more specific tissues or treatments. The sets of parameter values may suitably include one or more of the parameters PDC, BPW, PP, DCP, NOP, PW, AMPp, AMPpn, IBPI, pattern frequency, and an indication of whether the pattern is biphasic, monophasic, cathodic or anodic leading, as well as others. Each set of parameter values define a predetermine burst pulse pattern.

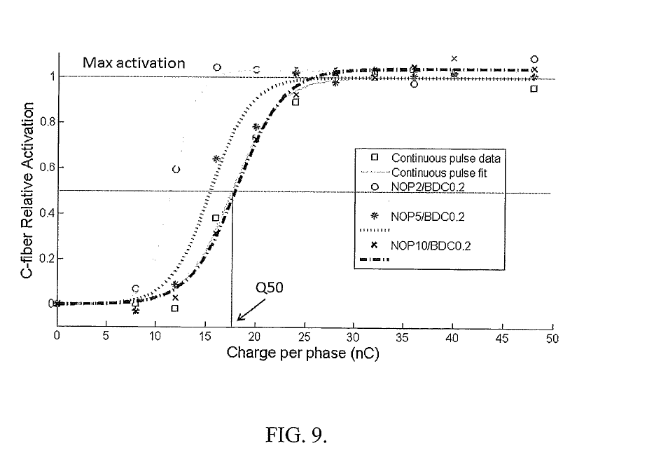

To this end, FIGS. 9-11 discuss testing of multiple waveforms comprising different burst pulse pattern waveforms on vagus and sciatic nerves in rats. From such tests, desirable burst pulse pattern parameters can be identified that are specific to each tissue and/or desired treatment effect. These sets of parameters can be stored, for example, in the memory 712 with an association to the tissue and/or treatment for which they are deemed advantageous.

Referring again to FIG. 7 specifically, it will be appreciated that the signal generation function 706 in the processing circuit 704 constitutes the signal generator 102 of FIG. 3, and is generally configurable to provide repeating burst pulse patterns such as those discussed above in connection with FIGS. 4, 5, 6A and 6B, using parameters received from the control function 708. The control function 708 in the processing circuit 704 constitutes the processing circuit 104 of FIG. 3, and is configured to provide control parameters to the signal generation function 706 by obtaining a set of parameters from the parameter data 714 that are associated with a select tissue or other treatment.

The signal generation function 706 is generally able to provide a digitized pulse signal to the digital-to-analog converter (D/A) 716. The D/A 716 is configured to generate an analog signal from the digitized pulse signal. The analog signal represents the repeating burst pulse pattern waveform such as any of those discussed above in connection with FIGS. 4, 5, 6A and 6B. The D/A 716 provides the signal to two outputs: the current pump 718 and the analog buffer 720. If a voltage-based pulse is desired, then the electrode(s) 106 would be coupled to the analog buffer 720. If a current-based pulse is desired, then the electrode(s) 106 would be coupled to the current pump 718.

FIG. 8 shows another embodiment of the signal generator 102 and the processing circuit 104 of FIG. 3. In FIG. 8, the processing circuit 104 is part of a general purpose computer 802. The signal generator 102 includes a pulse function generator 806, a current pump 818 and an analog buffer or amplifier 820.

In FIG. 8, the computer 802 includes a processing circuit 804 that executes program instructions to carry out a control function similar to the control function 708 of FIG. 7. The instructions or code 810 for the control function are stored in a memory 812 of the computer 802. Similar to the memory 712 of FIG. 7, the memory 812 may also suitably store sets of pulse and pattern parameters that correspond specifically to different tissues (e.g. nerves) to be stimulated. The sets of parameter values may suitably include one or more of the parameters PDC, BPW, PP, DCP, NOP, PW, AMPp, AMPpn, IBPI, pattern frequency, and an indication of whether the pattern is biphasic, monophasic, cathodic or anodic leading, as well as others.

It will be appreciated that the pulse function generator 806 may suitably be a standalone pulse signal generator or any other suitable device that is generally configurable to provide repeating burst pulse patterns such as those discussed above in connection with FIGS. 4, 5, 6A and 6B, using parameters received from the processing circuit 804.

The pulse function generator 806 is generally able to provide a repeating burst pulse pattern waveform such as any of those discussed above in connection with FIGS. 4, 5, 6A and 6B. The pulse function generator 806 in this embodiment provides an analog signal, and does not require any D/A conversion, except to the extent that the D/A is inherently included in the function generator 806 itself. In any event, the pulse function generator 806 provides the signal to two outputs: the current pump 818 and the analog buffer 820. If a voltage-based pulse is desired, then the electrode(s) 106 would be coupled to the analog buffer 820. If a current-based pulse is desired, then the electrode(s) 106 would be coupled to the current pump 818.

It will be appreciated that any existing waveform generation system can be used to create these burst modulated waveforms, assuming that the specifications are appropriate. Because these burst pulses can have especially short durations, it is important to ensure that all stages of the hardware are fast enough to follow the waveform, or else the final stimulus waveform will be distorted. Specifically, the signal generator 102 should be configured such that rise time of each burst pulse (e.g. pulse 206) should be well shorter than burst width.

It will be appreciated that either the analog buffer 720, 820 may be eliminated if there is no desire for voltage-based pulses Likewise, the current pump 718, 818 would not be necessary if there is no need for current pulses. However, it is widely accepted that current-controlled stimulation is more effective than voltage-controlled.

In the embodiment described herein, each of the current pumps 718, 818 may suitably comprise a Howland current pump. However, each of the pumps 718, 818 may alternatively be replaced by another suitable voltage to current conversion circuit or device. It is also imperative that the sampling rate of the D/A 716 is fast enough, based on frequency and pulse width of the burst pulses being generated.

One advantage of the embodiments described herein is that with higher frequency burst pulses, non-Faradaic charge transfer at the electrode 106 surface will occur more readily, and the effective impedance of the stimulation electrodes 106 will be lower than that when using conventional pulsing stimulation. From a device-tissue interface perspective, decreasing the effective impedance will advantageously enable the use of smaller stimulation electrodes, which have higher spatial selectivity, but tend to also have higher electrical impedances. Because the effective impedance is lower, the voltage and energy needed to drive the same amount of current or charge are advantageously reduced as well. Decreasing the amount of energy needed to inject the same amount of current or charge will reduce power consumption. In turn, reduced power consumption will prolong the battery life of battery-powered implants and make more feasible the implementation of wireless, battery-less implants, which have strict power constraints. Accordingly, at least one embodiment of the circuit in FIG. 3 is a battery-powered, implantable device.

In addition, because higher frequency bursts facilitate non-Faradaic charge transfer at the electrode surface, there is less potential for Faradaic charge transfer to occur. Reducing Faradaic charge transfer at the electrode surface will decrease oxidation-reduction reactions at the electrode-tissue interface and will lead to less damage to both the tissue and the electrode. As a result, this method will prolong the life of the implant and ensure the viability of the target tissue.

For neural stimulation-recording setups, the lower voltage needed to deliver electrical stimuli will produce a lower stimulus artifact. This feature reduces the required distance between the stimulating and recording electrodes and would allow researchers to experiment on smaller neural systems, e.g. a smaller, shorter nerve or a smaller animal. At the same time, artifact removal will be easier, yielding a cleaner desired neural recording.

Furthermore, burst modulation enables deeper tissue penetration and provides a vehicle for more effective, efficient, and safe charge delivery to a nerve or tissue. Chronic implants in neural tissue elicit an innate foreign body response that leads to device encapsulation by a reactive glial tissue that increases the impedance of the overall electrode-tissue system. The complex impedance of this reactive glial scar, as well as that for normal tissue, can be modeled with capacitors and resistors, and higher frequency stimuli will better penetrate both the glial scar and normal tissue.

While higher-frequency burst pulses have been used, the overall shape of the prior art pulse is still maintained as the shape of the pattern (e.g. 202, 204) in which the burst pulses occur. Therefore, stimulation using these burst modulated waveforms will remain in the same safety range as stimulation using conventional pulsing waveforms.

From a biological and physiological perspective, the bursting paradigm can be used to elicit a different response than that normally observed with conventional pulsing. Neural tissue is far from homogeneous, and different neuron populations have different energy requirements for activation and different activation kinetics. While keeping the pattern parameters (e.g. PW, AMPpn, IPI, pattern frequency) the same, the bursting parameters (e.g. PDC, BPW, PP, DCP, NOP, AMPp, IBPI) can be adjusted to more efficiently activate different neuron populations. As discussed above, the association of bursting parameters to select tissues, for example neuron populations, can be stored in a memory 712, 812. A user can then input to the processing circuit 704, 804 (via user interface equipment, not shown, but which may be conventionally coupled to the processing circuits 704, 804), an identification of a tissue, neuron population or the like. The processing circuit 704, 804 would then use the stored parameter data 714, 814 to obtain the proper burst pulse parameters, and control the signal generation functions 706, 806 to generate the periodic burst pulse pattern in accordance with those parameters.

In any event, the rapid, short burst pulses (e.g. pulses 206) within a pattern (e.g. 202a) have a duty cycle and strength sufficient to activate A-fiber types within a nerve (i.e., A-fiber types are activated with the 1st burst pulse), which then enter a refractory period for the remainder of the typical burst pulse duration (e.g., <1-2 ms). A-fiber types have shorter strength duration time constants, which in an electrical circuit analogy, is roughly equivalent to a capacitor that charges and discharges in a shorter time than a capacitor with a longer time constant. The fiber types with longer time constants—or in alternate terms, the fiber types whose membranes accumulate charge more slowly due to diffuse receptor distributions—will eventually reach an activation threshold if charge is accumulated more rapidly with each burst pulse than it is discharged. With low current, high duty cycle bursts, it is possible to select for B- and C-fibers using significantly less energy than conventional rectangular pulses. With high current, low duty cycle burst pulses, the same effect can be achieved in the A-fiber population.

### Experimental Results

Experimental results show that, with appropriate selection of parameters, burst modulated pulses (i.e. repeating burst pulse patterns such as those discussed above in connection with FIGS. 4, 5, 6A and 6B) can be constructed to favor efficacy, efficiency, as well selectivity.

For example, FIG. 9 illustrates charge-response curves of vagal C fibers resulting from vagus nerve stimulation in a rat. The data from a continuous (prior art) pulse waveform (parameters: 20 Hz pulse repetition, 1 s pulse train duration, 0.2 ms pulse width, at varying amplitudes) and from corresponding representative burst modulated pulse waveforms are shown. Each data point is an average of 20 stimuli with the same parameters. The lines show corresponding sigmoidal fit models (r² is generally >0.95). Activation levels are normalized to the maximal activation level achieved using a continuous rectangular pulse. For this particular nerve, the burst pulse pattern with the parameters, NOP=10, PDC=0.2 performed better in terms of efficacy (higher maximal activation). The burst pulse pattern with the parameters NOP=5, PDC=0.2 has better efficiency (lower Q50, the amount of charge needed to reach 50% activation). The burst pulse patter having NOP=2, PDC=0.2 is better in both.

Each waveform has its own distinctive curve. Compared to a continuous rectangular pulse waveform, burst modulated waveforms are capable of 1) eliciting stronger maximal response from the stimulated neural population (higher efficacy, see FIG. 9) reaching the same level of response with less charge per phase (better efficiency, see FIG. 9). The experiments testing these waveforms suggest that, in general, across different subjects, certain parameter combinations seem to produce waveforms with higher efficacy and/or efficiency. For the best results, however, test stimuli should be applied first to develop the charge-response curves and allow calibration.

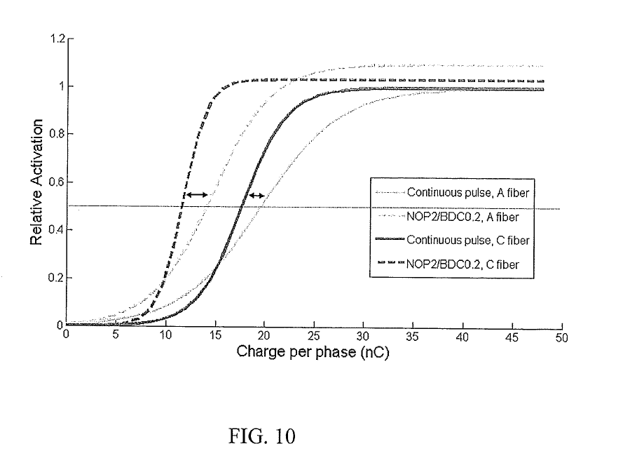

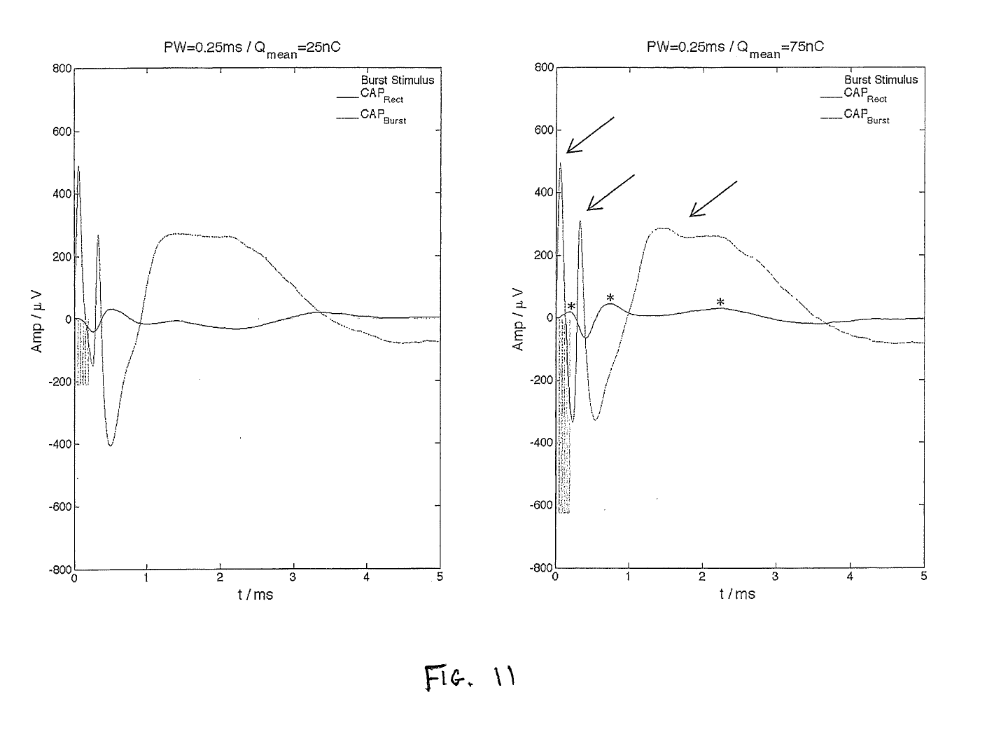

Also, importantly, the burst modulation parameters can be adjusted so as to better target one population compared to the other (more selectivity). In nerve stimulation, this effect can be clearly seen by comparing the efficiency of the waveforms at activating A and C fibers, as demonstrated in FIG. 10. In particular, FIG. 10 shows charge-response curves of vagal A and C fibers resulting from vagus nerve stimulation in rat. The figure shows the sigmoidal fit models from a continuous rectangular pulse waveform (parameters: 20 Hz pulse repetition, 1 s pulse train duration, 0.2 ms pulse width, at varying amplitudes) and from a corresponding NOP=2, PDC=0.2 BDC burst modulated pulse waveform. Activation levels of both A and C fibers are normalized to the maximal activation level achieved using a continuous pulse. Based the Q50 information, a measure of efficiency, the burst modulated pulse is more efficient at activating C fibers than A fibers, when compared to the continuous pulse.

When electrically stimulating excitable tissue, the cells nearest to the electrode are expected to respond first. To this end, it takes time for the injected charges to flow through the tissue. Because the impedance of electrical charge flowing through tissue tends to decrease as the frequency of the stimulus increases, using burst modulated pulses can allow the injected charges to flow through the tissue faster, so that the cells in range will respond faster. In nerve stimulation, this effect manifests as 1) lower peak latencies as well as 2) shorter peak widths. The faster response and increased synchrony can have significant impact on physiology and therapy.

By way of illustration of this advantage, FIG. 11 shows compound action potential (CAP) recordings resulting from stimulation of the sciatic nerve in a rat. The CAP_{rect} trace is the recording from conventional, continuous rectangular pulsing, while the CAP_{burst} is the recording from burst modulated pulsing. The burst modulated stimulus is also included for reference. Two trials are shown: the left a smaller amplitude, with mean charge injected=25 nC; the right with mean charge injected=75 nC. In either trial, the difference in the two CAP responses is obvious. Furthermore, the peaks resulting from burst modulated pulses appear closer to the onset of the stimulus (lower latency) and have shorter peak widths (please note that the peaks from the continuous pulse tend to be wider when they are more apparent).

It will be appreciated that that the above-described embodiments are merely illustrative, and that those of ordinary skill in the art may readily devise their own implementations and modifications that incorporate the principles of the present invention and fall within the spirit and scope thereof.

What is claimed is:

1. A method for stimulating nerve tissue of an organism, comprising:
a) electrically connecting an electrical signal source to a first nerve;
b) applying a periodically repeating burst pulse signal pattern to the first nerve, the burst pulse signal pattern having a pattern frequency defining a frequency of repetition of the burst signal pattern and a pattern duty cycle defining a first time period of the burst pulse signal pattern, the burst pulse signal pattern having a plurality of pulses, the pulses having a frequency within the pattern that exceeds the pattern frequency by at least an order of magnitude.

2. The method of claim 1, wherein the pulses within the burst pulse signal pattern have a constant magnitude.

3. The method of claim 1, wherein the pulses within the burst pulse signal pattern define a sequence, wherein peak magnitudes of the pulses within the sequence define at least a portion of a sine wave.

4. The method of claim 1, wherein the pulses within the burst pulse signal pattern define a sequence, wherein peak magnitudes of the pulses within the sequence define at least a portion of a triangular wave.

5. The method of claim 1, wherein the pulses within the burst pulse signal pattern define a sequence, wherein peak magnitudes of the pulses within the sequence define at least a portion of a Gaussian wave pattern.

6. The method of claim 1, wherein step b) further comprises applying a steady state signal during a second time period between successive burst pulse signal patterns.

7. The method of claim 1, further comprising
c) applying a second burst pulse signal pattern during a second time period between successive burst pulse signal patterns.

8. The method of claim 1, wherein an average signal magnitude of the burst pulse signal pattern and a second average magnitude of the second burst pulse signal pattern define a biphasic pulse signal pattern having the pattern frequency.

9. A method for stimulating nerve tissue of an organism, comprising:
a) electrically connecting an electrical signal source to at least a first nerve;
b) applying a periodically repeating burst pulse signal pattern to the first nerve, the burst pulse signal pattern having a pattern frequency defining a frequency of repetition of the burst pulse signal pattern and a pattern duty cycle defining a first time period of the burst pulse signal pattern, the burst pulse signal pattern having a plurality of pulses, wherein applying the periodically repeating burst pulse signal pattern includes applying a select burst signal pattern corresponding to a type of the first nerve.

10. The method of claim 9, wherein the select burst signal pattern has one of plurality of predefined burst pulse amplitudes, the one of the plurality of predefined burst pulse amplitudes corresponding to the type of the first nerve.

11. The method of claim 9, wherein the select burst signal pattern has one of plurality of predefined burst pulse periods, the one of the plurality of predefined burst pulse periods corresponding to the type of the first nerve.

12. The method of claim 9, wherein the select burst signal pattern has one of plurality of predefined burst pulse widths, the one of the plurality of predefined burst pulse widths corresponding to the type of the first nerve.

13. The method of claim 9, wherein the select burst signal pattern has one of plurality of predefined inter-burst intervals, the one of the plurality of predefined inter-burst intervals corresponding to the type of the first nerve.

14. A system, comprising:
a signal generator;
at least one electrode;
a processing circuit operably coupled to a signal generator, the processing circuit configured to cause the signal generator to generate a periodically repeating burst pulse signal pattern to the first nerve, the burst pulse signal pattern having a pattern frequency defining a frequency of repetition of the burst signal pattern and a pattern duty cycle defining a first time period of the burst pulse signal pattern, the burst pulse signal pattern having a plurality of burst pulses, the burst pulses having a frequency that exceeds the first pulse frequency by at least an order of magnitude.

15. The system of claim 14 further comprising a memory storing a plurality of sets of burst pulse parameters, each defining one of a plurality of burst signal patterns.
